(19) United States
(12) Patent Application Publication
(10) Pub. No.: US 2002/0197614 A1
(43) Pub. Date: Dec. 26, 2002

(54) Electrophoretic analysis of target molecules using adapter molecules

(75) Inventors: Lawrence Weir, Hopkinton, MA (US); T. Christian Boles, Waltham, MA (US); Ezra S. Arams, Newton, MA (US)

Correspondence Address: Patent Administrator, Testa, Hurwitz & Thibeault, LLP, High Street Tower, 125 High Street, Boston, MA, 02110, US

(73) Assignee: Mosaic Technologies, Inc., Waltham, MA

(21) Appl. No.: 09/968,084

(22) Filed: Oct. 1, 2001

Related U.S. Application Data
(63) Continuation of application No. PCT/US00/08529, filed on Mar. 31, 2000.
Application No. PCT/US00/08529 is a continuation of application No. 09/285,380, filed on Apr. 2, 1999, now abandoned.
Application No. 09/285,380 is a continuation-in-part of application No. 08/971,845, filed on Aug. 8, 1997.
(60) Provisional application No. 60/046,708, filed on May 16, 1997.

(51) Int. Cl. C12Q 1/68; C12M 1/34

(52) U.S. Cl. 435/6; 435/287.2; 204/461

(57) ABSTRACT

A universal gel and methods of use in detecting the presence, or absence, of one or more target molecules in a test sample comprising the formation of a universal gel hybridization complex, wherein an adapter molecule is hybridized to a target molecule and universal capture probe is disclosed.

### RELATED APPLICATIONS

This application is a continuation of PCT/US00/08529, filed Mar. 31, 2000, which claims priority to U.S. application Ser. No. 09/285,380, filed Apr. 2, 1999, which is a continuation in part of U.S. application Ser. No. 08/971,845, filed Aug. 8, 1997, which claims the benefit of Provisional Application No. 60/046,708, filed May 16, 1997, the entire teachings of which are incorporated herein by reference.

### BACKGROUND OF THE INVENTION

Nucleic acid base pairing is an extremely high affinity and specific interaction. For this reason, nucleic acid hybridization assays have been devised for a variety of diagnostic purposes.

Under laboratory conditions, hybridization assays can be extraordinarily sensitive, detecting femtogram amounts of a specific molecule. However, several technical limitations have prevented widespread use of hybridization analysis in commercial diagnostic techniques.

First, use of high activity hybridization probes requires stringent procedures for separating unhybridized (or improperly hybridized) and hybridized probe. This separation can be facilitated by the use of solid phase hybridization formats, in which either the sample nucleic acid or the probe that is complementary to the desired target is immobilized on a solid support. Hybridized and unhybridized species can be separated by washing the support.

A second limitation of hybridization assays is that efficient hybridization of samples containing low concentrations of target nucleic acids frequently requires lengthy incubations (up to several hours) under carefully controlled conditions. Unfortunately, use of solid phase assays exacerbates this problem, since immobilized nucleic acids virtually always hybridize with slower kinetics than nonimmobilized ones.

For these reasons, a number of workers have sought methods to perform solid phase hybridizations with better kinetics and efficiency. Several groups have found that inclusion of high molecular weight polymers such as dextran sulfate or polyethylene glycol improves solid phase assay performance, albeit modestly. (Wieder and Wetmur, Biopolymers, 20:1537 (1981); Wetmur, Biopolymers, 14:2517 (1975); Yokota and Oishi, Proc. Natl. Acad Sci. USA, 87:6398 (1990)). Several groups have developed chromatographic solid phase hybridization methods that show improvements. In general, it has been found that flowing the solution phase nucleic acid strand over (or through) the solid support bearing the immobilized strand improves both kinetics and efficiency of hybridization. MacMahon and Gordon, U.S. Pat. No. 5,310,650, describes immobilized target molecules on nitrocellulose filters, with labeled probe flowing through the immobilized target regions by capillary action. In a similar experiment, Reinhartz et al. (Gene, 136:221-226 (1993)) immobilized capture probes on paper filters and flowed labeled single-stranded PCR products through the capture probe region, again using capillary action. Others have demonstrated improved hybridization assays by passing samples through an HPLC column containing silica particles covalently modified with capture probes. (Tsurui et al., Gene, 88:233-239 (1990)). Typically, these solid phase methods can only be employed for predetermined target molecules and must be constructed de novo. Therefore, their versatility as hybridization gels is limited to a specific assay because the gel itself is dedicated to the capture of predetermined target molecules.

There exists a need for a method of solid phase hybridization that has universal utility, that is, it has the ability to be employed in the capture of any and all target molecules amenable to hybridization in an electrophoretic medium.

### SUMMARY OF THE INVENTION

The present invention relates to the discovery that nucleic acids, modified nucleic acids and nucleic acid analogs can be immobilized (e.g., covalently attached) to an electrophoretic medium and that electrophoresis can be used to separate, purify or analyze target molecules that specifically bind to (e.g., associate with), or are specifically bound by, the immobilized nucleic acids, modified nucleic acids or nucleic acid analogs. The immobilized nucleic acids, modified nucleic acids or nucleic acid analogs, are referred to herein as universal capture probes (also referred to herein as “capture probes”). Typically these capture probes are specific for only one class of adapter molecule. Therefore, the gel which possesses a specific class of capture probe, or probes, can only be used for that specific molecule from which the capture probe has been designed.

The present invention specifically relates to a solid phase hybridization system for detecting the presence or absence of a target molecule based on a “universal capture” electrophoresis gel. The universal capture gel (also referred to herein as “universal gel”) comprises an electrophoretic medium (also referred to herein as an electrophoretic matrix) within which a capture probe is immobilized. More specifically, the universal capture gel described herein is an electrophoretic gel comprising universal capture probes copolymerized within the gel. The capture probes can be immobilized throughout the gel, or in discrete layers of the gel (e.g., forming capture layers). The universal capture gel can be pre-cast in a variety of formats such as a slab gel or capillary gel and prepared and stored prior to use.

The universal capture gel system of the present invention has three components. The first component is a universal capture probe. The capture probe comprises a nucleotide sequence region which is complementary to an adapter molecule. The capture probe is a nucleic acid molecule, either DNA or RNA, usually from about 18 to about 24 nucleotides in length, which is immobilized within the gel by, for example, covalent attachment. The second component is an adapter molecule. The adapter molecule is typically a nucleic acid molecule usually from about 15 to about 40 nucleotides in length. The adapter molecule usually comprises two nucleotide sequences joined together to form two nucleotide sequence regions as described below. The third component is an electrophoretic medium suitable for performing electrophoresis, for example, an acrylamide gel.

A key component of the present invention is the adapter molecule. An adapter molecule encompassed by the present invention has at least two nucleotide sequence regions. One region is complementary to a capture probe nucleotide sequence, and, therefore, specifically hybridizes with the capture probe. This region is referred to herein as the “capture probe-specific” nucleotide sequence region. The other region is complementary to a target molecule nucleotide sequence (e.g., bacterial rRNA), and, therefore, specifically hybridizes with a nucleotide sequence region contained within a target molecule. This region is referred to herein as the “target-specific” nucleotide sequence region. The universal capture gel of the present invention is constructed to have at least one class of capture probes immobilized within the electrophoretic medium which can hybridize with any adapter molecule having a nucleotide sequence region complementary to the capture probe. In one embodiment of the present invention, multiple discrete regions within the electrophoretic medium contains separate classes of capture probes, and adapter molecules that are specific for a particular class are used such that multiple target molecules can be captured and isolated within these discrete regions of the electrophoretic medium. Adapter molecules can be designed to analyze any target molecules comprising a nucleotide sequence contained within a test sample, for example, bacterial, viral, fungal, plant, animal (including, but not limited to, vertebrates like mammals such as human) target molecules and combinations thereof.

Also encompassed by the present invention are universal gel hybridization complexes comprising an adapter molecule hybridized to a target molecule. The adapter molecule comprises a capture probe-specific nucleotide sequence region which is complementary to a capture probe, and a target-specific nucleotide sequence region which is complementary to a target molecule. This adapter/target hybridization complex described herein is typically formed in a solution phase prior to introduction into the universal capture gel.

The present invention further encompasses methods of using the universal capture gel described herein. In one embodiment of the invention, a method of detecting the presence, or absence, of a target molecule in a test sample using a universal capture gel is described. A universal capture gel is employed comprising one class of capture probes immobilized throughout the electrophoresis medium. A test sample which is being analyzed for the presence, or absence, of a target molecule is contacted with (e.g., mixed with) an adapter molecule that has at least one nucleotide sequence region that is complementary to the target molecule to be detected. An adapter/target complex (also referred to herein as a universal gel hybridization complex) is formed under conditions suitable for the adapter molecule's nucleotide sequence region specific for the target molecule to hybridize with the target molecule and form a stable complex. This adapter/target complex is subjected to electrophoresis through the universal capture gel. The complex migrates through the gel until the complex contacts an immobilized capture probe specific for the adapter molecule. Once the adapter/target complex and complementary capture probe hybridize, a tripartite hybridization complex is formed comprising the capture probe and the adapter/target complex, which is immobilized within the universal capture gel. The detection of this immobilized tripartite complex is indicative of the presence of the target molecule in the original test sample.

In another embodiment, a universal capture gel with one class of immobilized capture probes is used for detecting one, or more, target molecules in a test sample. In this embodiment it may not be desirable to identify (e.g., detect specifically) the different target molecules per se, it may only be desirable to qualitatively detect the presence of any target molecule contained within a test sample. Multiple classes of adapter molecules are used in which they all share one nucleotide sequence region that is complementary to the capture probe immobilized in the electrophoretic medium, but differ with respect to their target nucleotide sequence complementarity region. A test sample which is being analyzed for the presence, or absence, of a target molecule is contacted with (e.g., mixed with) an adapter molecule that has at least one nucleotide sequence region that is complementary to the target molecule to be detected. A universal gel hybridization complex is formed under conditions suitable for the adapter molecule's nucleotide sequence region specific for the target molecule to hybridize with the target molecule and form a stable complex. This adapter/target complex is subjected to electrophoresis through the universal capture gel. The complex migrates through the gel until the complex contacts an immobilized capture probe specific for the adapter molecule. Once the adapter/target complex and complementary capture probe hybridize, a tripartite hybridization complex is formed comprising the capture probe and the adapter/target complex, which is immobilized within the universal capture gel. The detection of this immobilized tripartite complex is indicative of the presence of the target molecule in the original test sample.

In still another embodiment, it may be desirable to specifically detect different target molecules using the universal capture gel. In this embodiment multiple classes of capture probes are immobilized within the electrophoretic medium in discrete regions, with each discrete region possessing only one class of capture probe comprising a nucleotide sequence complementary to nucleotide sequence of a specific class of adapter molecule. Each class of adapter molecule in turn comprises a nucleotide sequence complementary to a specific target molecule, or class of target molecule (e.g., rRNA common to bacteria). The adapter molecules and target molecules are contacted with one another under conditions suitable for hybridization. The different adapter/target complexes are then subjected to electrophoresis through the universal capture gel. When the individual adapter/target complex comes in contact with an appropriate immobilized capture probe, then the complex becomes hybridizes to the capture probe forming a tripartite hybridization complex which is immobilized within the gel.

By changing a single component of an adapter molecule (i.e., by changing the target-specific nucleotide region of the adapter molecule), the capture and detection of any DNA or RNA can be achieved using the universal capture gel format described herein. Thus, as a result of the work described herein, a universal capture gel and methods of its use are now available for fast, efficient and accurate electrophoretic analysis of target molecules using universal capture gels comprising immobilized capture probes that specifically bind to adapter/target complexes.

In another embodiment of the present invention, a method for purifying at least one target molecule from a test sample is disclosed. A test sample from which at least one target molecule is to be purified is contacted with (e.g., mixed with) an adapter molecule that has at least one nucleotide sequence region that is complementary to the target molecule to be detected. A universal gel hybridization complex is formed under conditions suitable for the adapter molecule's nucleotide sequence region specific for the target molecule to hybridize with the target molecule and form a stable complex. This adapter/target complex is subjected to electrophoresis through the universal capture gel. The complex migrates through the gel until the complex contacts an immobilized capture probe specific for the adapter molecule. Once the adapter/target complex and complementary capture probe hybridize, a tripartite hybridization complex is formed comprising the capture probe and the adapter/target complex, which is immobilized within the universal capture gel. A modified adapter molecule can be used to displace either the target molecule alone, or the target/adapter complex from the immobilized capture probe.

### BRIEF DESCRIPTION OF THE FIGURES

FIG. 1 is a schematic representation of a capture complex being comprised of an immobilized capture probe, adapter molecule and target molecule.

FIG. 2 is a photograph of a gel showing the results of an experiment using the universal capture gel to detect two different RNA target molecules.

FIG. 3 is an schematic representation of myosin mRNA capture using an adapter molecule.

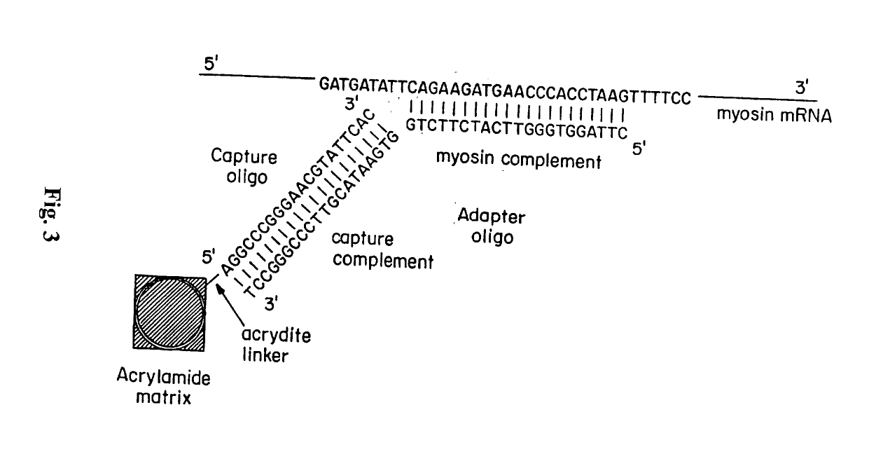

FIG. 4 is the nucleotide sequence of RNA 1.

FIG. 5 is the nucleotide sequence of RNA 2.

### DETAILED DESCRIPTION OF THE INVENTION

The present invention relates to a universal capture gel system. This universal capture gel system is comprised of three components. The first component of the universal capture gel system is a universal capture probe (or simply, capture probe). The capture probe is a short sequence of a nucleic acid, modified nucleic acid or nucleic acid analog, usually about 5 to about 100 nucleotides in length, which is immobilized within an electrophoretic medium and together, they form a universal gel. The capture probes can be immobilized within a discrete layer, or layers, of the gel forming one or more capture layers. Alternatively, the capture probes can be immobilized throughout the electrophoretic medium. The capture probe hybridizes with an adapter molecule that comprises at least one nucleotide sequence region that is complementary to the capture probe nucleotide sequence. Therefore, a heterogeneous set of adapter molecules specific for different target molecules can all bind to just one class of capture probe, if the heterogenous set of adapter molecules share at least one complementary nucleotide sequence region to a single class of capture probes.

The capture probe typically forms a stable complex with an adapter molecule. A “stable complex” as defined herein is a complex with a bound lifetime that is long in comparison to the duration of the electrophoresis. The appropriate length and sequence of the capture probe and adapter necessary to achieve adequate stability is a function of the chemical nature of the adapter and capture probe, the length and sequence of the potential duplex formed by them, and the conditions of electrophoresis. For example, for DNA adapter and capture probes, using electrophoresis in 1× TBE (89 mM Tris-borate, pH 8.3, 2 mM EDTA) at 25° C. and 10 V/cm, the minimum useful duplex length is around 15 base pairs. However, using 2′-O-methyl capture probes and RNA adapters, the minimum useful duplex length is around 9 base pairs. If PNA capture probes are used with DNA adapters, then even shorter duplexes are stable, although the stability is extremely sequence-dependent for such short duplexes. Methods for designing a capture probe-adapter combination with defined levels of stability are well known in the literature (Wertmur, Critical Reviews in Biochemistry and Molecular Biology, 26:227-259 (1991); Wertmur and Sninsky, in PCR Strategies, Innis, M. A., et al., (eds). Academic Press, San Diego, Calif., ch 6, pp. 69-83 (1995); Freier and Altman, Nucleic Acid Res., 25:4429-43 (1997); U.S. Ser. No. 09/188,086.

The second component of the universal capture gel system is the adapter molecule. The adapter molecule is typically a polynucleotide from about 10 to about 100 nucleotides in length and comprises two nucleotide sequence regions. The two nucleotide sequence regions can be immediately adjacent to each other or can have a short region of intervening nucleotides between the two regions. The two regions can comprise subregions within one larger nucleic acid. The two regions within the adapter can be contiguous or non-contiguous within a larger nucleic acid molecule. The two regions can have different chemical compositions or modifications. For example, one region can be DNA and the other region can be PNA. One region is a capture probe-specific nucleotide sequence region complementary to a nucleotide sequence region of the capture probe. Another region is a target-specific nucleotide sequence region complementary to a nucleotide sequence region of the target molecule. Thus, the adapter molecule of the present invention specifically hybridizes with both a capture probe and a target molecule.

The third component of the universal capture gel comprises one, or more, capture probes, or classes of capture probes, immobilized in a matrix suitable for electrophoresis.

Sample

The test sample can be from any source containing nucleic acids and can contain any molecule comprising a nucleotide sequence that can form a hybridization complex with a capture probe. Specifically encompassed by the present invention are samples from biological sources containing cells, obtained using known techniques, from body tissue (e.g., skin, hair, internal organs), body fluids (e.g., blood (e.g., platelets, erythrocytes), plasma, urine, semen, sweat) or cell and/or tissue culture systems. Other sources of samples suitable for analysis by the methods of the present invention are microbiological samples, such as bacteria, viruses and yeasts, plasmids, isolated nucleic acids and agricultural or food samples, such as recombinant plants and plant cells.

Sample Preparation

The test sample is treated in such a manner, known to those of ordinary skill in the art, so as to render the target molecules contained in the test sample available for hybridization. For example, if the target molecule is a nucleic acid present in a bacterial cell, a bacterial cell lysate is prepared, and a crude bacterial cell lysate (e.g., containing the target nucleic acid as well as other cellular components such as proteins and lipids) can be analyzed. Alternatively, the target nucleic acids can be isolated (rendering the target nucleic acids substantially free from other cellular components) prior to analysis. Isolation can be accomplished using known laboratory techniques. The target nucleic acid can also be amplified (e.g., by polymerase chain reaction or ligase chain reaction techniques) prior to analysis.

A suitable sample preparation involves steps taken to lyse the cell or cells, thereby releasing their nucleic acids. For a bacterial target, preferably the RNA is liberated from the constraints of the cell wall and cell membrane. If the target is a viral, fungal, parasitic, plant or animal molecule, either the DNA or RNA can be a target molecule and must be released from the interior compartments of the organism. Optionally, additional steps can be taken in order to further purify the target nucleic acid desired from contaminating molecules, such as cellular debris after lysis. Methods of purifying nucleic acids are well known to those of ordinary skill in the art. (Ausubel, F. M., et al., (eds), Current Protocols in Molecular Biology, John Wiley & Sons (Pub.), vol. 1, ch. 2 through 4 (1991), the teachings of which are herein incorporated by reference in its entirety).

Target Molecule

In one embodiment of the present invention, a single-stranded target molecule, a single-stranded adapter molecule and a single-stranded immobilized probe are used. This embodiment is especially useful for analysis of RNA targets. It is also useful for capture of specific targets from complex samples where renaturation of the target is not rapid. Highly concentrated targets, such as PCR products, may require denaturation immediately prior to electrophoresis because of rapid renaturation. For example, for analysis of PCR products that are 100-250 base pairs in length, it is convenient to bring the sample to 75% formamide (volume/volume) and heat at greater than 75° for 5 minutes immediately prior to electrophoresis. Target molecules can have a size of from about 10 to about 100,000 nucleotides in length.

In another embodiment of the present invention, a double-stranded target is placed in a complex with a single-stranded adapter molecule. For example, adapter molecules can be designed that will associate with double-stranded nucleic acids to form a triple-stranded structure. The third strand locates in the major groove of the duplex and forms Hoogsteen base pairing interactions with the bases of the duplex (Hogan and Kessler, U.S. Pat. No. 5,176,966 and Cantor, et al., U.S. Pat. No. 5,482,836). The design of the adapter molecule is therefore subject to the constraints governing those chemical interactions. However, the frequency of sequences capable of forming triplex structures in naturally occurring nucleic acids is high enough that many target nucleic acids can be specifically captured using this adapter molecule design strategy.

Alternatively, adapter molecules can be designed that will associate with double-stranded nucleic acids by formation of a displacement loop structure. Such adapter molecules bind to only one strand of the duplex nucleic acid and displace the adapter polynucleoide-homologous duplex strand of the duplex locally. This displacement can only be achieved if the adapter molecule-target strand interaction is much more favorable than the interaction between the target strands. Such adapter molecules can be made using modified bases and techniques described in Wetmur, Critical Reviews in Biochemistry and Molecular Biology, vol 26, pp 227-259 (1991), backbone modifications (Moody, et al., Nucleic Acids Res., vol. 17, pp. 4769-4782 (1989)) and nucleic acid analogs (Nielson, et al., Science, 254:1497-1500 (1991)). The use of peptide nucleic acid (PNA) probes which base pair exceptionally tightly and specifically with naturally occurring nucleic acids would also be useful in this embodiment.

Adapter Molecules

A variety of adapter molecules can be used in the methods disclosed by the present invention. The adapter molecule is typically from about 10 to about 100 nucleotides in length. Preferably, the adapter molecule contains from about 10 to about 50 nucleotides. An adapter molecule comprises at least two distinct nucleotide sequence regions. The capture probe-specific nucleotide sequence region, from about 5 to about 50 nucleotides in length, is complementary to a nucleotide sequence defined by the target molecule desired to be captured. The target-specific nucleotide sequence region, from about 5 to about 50 nucleotides in length, of the adapter molecule is complementary to a nucleotide sequence contained within a capture probe. The length of each region of the adapter depends upon the relative base pairing strength of the specific nucleic acids used. For example, considering identical duplexes of different chemical composition, the relative duplex stabilities will be DNA:DNA=2′-O-methyl RNA:DNA<DNA:RNA<2′-O-methyl RNA:RNA<PNA:DNA<PNA:RNA. Thus, the length of duplex needed for stable DNA:DNA duplex formation is significantly greater than that for a PNA:DNA duplex. Preferably, the adapter is synthesized using standard automated methods for oligonucleotide or peptide (for PNA) synthesis wherein both sequence regions form part of a single longer adapter molecule. Alternatively, the two sequence regions can be synthesized separately and subsequently linked using chemical crosslinkers well known to those skilled in the art. (Wong, Chemistry of Protein Conjugation and Cross-linking, CRC Press, Boca Raton, Fla. (1991)). The latter strategy can be especially useful for chimeric adapters where the two regions are chemically different, that is, a PNA capture probe-specific domain with a DNA target-specific domain. Typically, the adapter molecule is a DNA molecule. However, the adapter molecule can be an RNA molecule. The adapter molecule can be either a single, double, or partially double stranded nucleic acid. Preferably, the adapter molecule is a single-stranded nucleic acid.

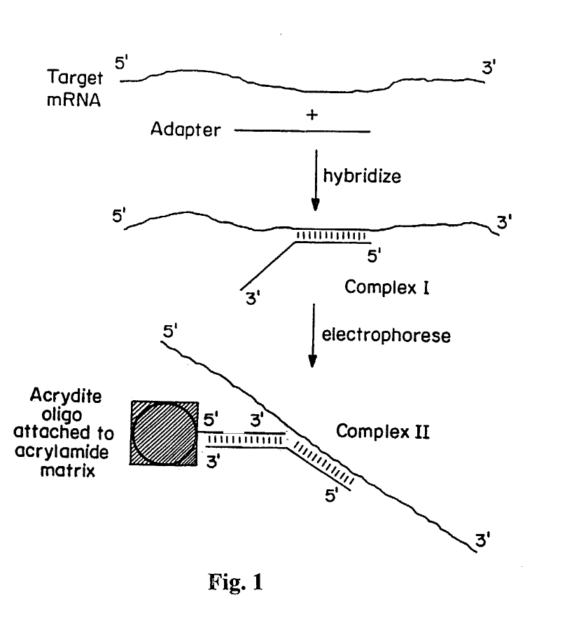

The adapter molecule brings together in a hybridization complex the target molecule and the capture probe, that is, it serves as a connector between the target molecule and capture probe, as shown in FIG. 1. Only target molecules with nucleotide sequence complementary to all, or a portion, of the nucleotide sequence of the adapter molecule will be captured during electrophoresis through the universal capture gel. Any contaminating molecule (e.g., a molecule that does not comprise a nucleotide sequence complementary to the adapter molecule) will migrate through the capture layer of the gel and will go undetected. Hence, this adapter technology can also be useful in purification schemes.

The adapter molecule allows for versatility with respect to the adapter/capture probe complex. The same capture probe can be used to detect multitude of test molecules because of the adapter molecule. The nucleotide sequence region of the adapter molecule that hybridizes to a particular capture probe will remain constant (i.e., a non-variable nucleotide sequence), while the nucleotide sequence region of the adapter molecule that hybridizes to a particular target molecule can vary (i.e., a variable nucleotide sequence) depending upon the specific target molecule desired to be captured. Therefore, a set of adapter molecules can be produced which have the ability to bind to the same capture probe, while possessing specificity for different target molecules. Therefore, only one class of capture probes is required in order to detect one, or many, target molecules using a universal capture gel. Moreover, only one type of electrophoretic medium need be produced for analyzing multiple target molecules independently given that the same class of capture probes can be used for a variety of target molecules. For example, if there are ten target molecules to be analyzed the practitioner can use ten electrophoretic gels that are the same, that is, contain the same capture probes immobilized within the gel. It is the adapter molecule which provides for this versatility of target molecule analysis which can employ only one type of gel comprising one class of capture probes.

Adapter/Target Hybridization

The target molecule from the test sample is brought in contact with an appropriate adapter molecule in solution under conditions suitable for hybridization and the target molecule of a test sample will hybridize with the adapter molecule forming a universal gel hybridization complex (i.e., adapter/target complex). For example, hybridization can be achieved by heating the solution containing the adapter molecule and target molecule to 90° C. and allowing to cool to room temperature.

Hybridization of adapter molecule to a target molecule can be accomplished in several ways. First, target and adapter molecules can be hybridized together prior to electrophoresis. For example, an RNA target is mixed in solution with a DNA adapter molecule (adapter concentration is form about 0.05 micromolar to about 1 micromolar) in from about 0.05 M to about 1 M monovalent salt, heated to around 90° C. and allowed to slow cool (over from about 30 minutes to about 1 hour) down to room temperature.

Second, the adapter can be hybridized to the capture probe prior to loading the target on the universal gel. In this embodiment, an adapter molecule is loaded in about 2-fold molar excess over the amount of the capture probe immobilized within the gel (or gel lane) and subjected to electrophoresis through the universal gel under conditions suitable for adapter hybridizing to the immobilized capture probe. The capture probes, therefore, become saturated with adapters, and remain stably immobilized on the capture layer. The target molecules can then be loaded onto the gel and subjected to electrophoresis through the capture layer where they can hybridize to the single-stranded target-specific region of the adapter molecule. In effect, pre-electrophoresis of the universal gel with an excess of adapter changes the specificity of the capture layer from a an adapter-specific sequence to a target-specific sequence. If different adapters are pre-electrophoresed in different lanes of the gel, the same universal capture layer can have different target specificities in adjacent lanes.

Third, hybridization between adapters and targets can occur in the gel during electrophoresis using sequential loading of target and adapter. Typically, the target has lower electrophoretic mobility than the adapter. If the target is loaded before the adapter molecule, then a concentrated layer of adapter will overtake and pass the slower moving target during electrophoresis. As the adapters pass the target band, they can hybridize with the target. Subsequently, the target-adapter complexes can be captured on a universal capture layer.

Single and Double Stranded Adapter Molecules and Targets for Analysis of Nucleic Acid Binding Proteins

The methods of the present invention are also useful for analysis of nucleic acid binding proteins. In these cases, the adapter molecules that are selected mimic, in some manner, the protein's natural binding substrate.

Both sequence-specific and non-sequence-specific nucleic acid binding proteins can be analyzed. For analysis of sequence-specific binding proteins, the adapter molecule is designed to contain a sequence which is recognized by the target binding protein. For analysis of non-specific interactions, mixtures of adapter molecules can be used to ensure that any observed binding is not dependent on any particular nucleic acid sequence.

Electrophoretic analysis is performed under conditions which allow the protein to retain its native structure, thereby permitting the protein to bind to the adapter molecule during electrophoresis. Following electrophoresis, the presence of the protein within the gel region containing an immobilized capture probe specific for the adapter molecule (where the adapter/target complex binds) can be detected by staining with colored or fluorescent dyes, autoradiography (if the sample has been radioactively labeled), silver staining, as well as other various other standard methods well known to those of ordinary skill in the art of protein electrophoresis.

For detection and analysis of sequence-specific DNA binding proteins that are important in transcriptional regulation, it is particularly useful to utilize double-stranded adapter molecules. In this implementation, a double-stranded adapter molecule (or partially double-stranded) containing a sequence known (or suspected) to be recognized by the protein target is used. The test sample is subjected to electrophoresis through the region containing a capture probe specific for the adapter molecule. Following electrophoresis, the position of the protein within the gel is determined. The presence of protein in the gel region containing the adapter/target/capture probe complex indicates the presence of a DNA binding-protein in the sample. Control experiments demonstrating that binding does not occur with a DNA adapter molecule, which lacks the specific sequence of interest, can be used to demonstrate the sequence specificity of the binding.

Single stranded adapter molecules may also be useful. For instance, single-stranded RNA adapter molecule can be used for detection and purification of proteins that bind to specific RNA sequences. Single-stranded DNA adapter molecules may be useful for detecting regulatory proteins of viruses that contain single-stranded DNA genomes, or proteins that bind specifically to single-stranded DNA segments within replication origins.

Electrophoretic Matrices

Any matrix suitable for electrophoresis can be used for the methods of the present invention. Suitable matrices include acrylamide and agarose, both commonly used for nucleic acid electrophoresis. However, other materials may be used as well. Examples include chemically modified acrylamides, starch, dextrans and cellulose-based polymers. Additional examples include modified acrylamides and acrylate esters (for examples see Polysciences, Inc., Polymer & Monomer catalog, 1996-1997, Warrington, Pa.), starch (Smithies, Biochem. J., 71:585 (1959); product number S5651, Sigma Chemical Co., St. Louis, Mo.), dextrans (for examples see Polysciences, Inc., Polymer & Monomer Catalog, 1996-1997, Warrington, Pa.), and cellulose-based polymers (for examples see Quesada, Current Opin. in Biotechnology, 8:82-93 (1997)). Any of these polymers listed above can be chemically modified to allow specific attachment of capture probes for use in the present invention.

Specifically encompassed by the present invention is the use of nucleic acids, modified nucleic acids or nucleic acid analogs as capture probes. Methods of coupling nucleic acids to create nucleic acid-containing gels are known to those of ordinary skill in the art. Nucleic acids, modified nucleic acids and nucleic acid analogs can be coupled to agarose, dextrans, cellulose, and starch polymers using cyanogen bromide or cyanuric chloride activation. Polymers containing carboxyl groups can be coupled to synthetic capture probes having primary amine groups using carbodiimide coupling. Polymers carrying primary amines can be coupled to amine-containing probes with glutaraldehyde or cyanuric chloride. Many polymers can be modified with thiol-reactive groups which can be coupled to thiol-containing synthetic probes, other suitable methods can be found in the literature. (For review see Wong, “Chemistry of Protein Conjugation and Cross-linking”, CRC Press, Boca Raton, Fla., 1993).

Methods for covalently attaching the capture probes described herein to polymerizable chemical groups have also been developed. When copolymerized with suitable mixtures of polymerizable monomer compounds, matrices containing high concentrations of immobilized nucleic acids can be produced. Examples of methods for covalently attaching nucleic acids to polymerizable chemical groups are found in U.S. Ser. No. 08/812,105, entitled “Nucleic Acid-Containing Polymerizable Complex,” and Rehman et al., Nucleic Acid Res., 27:649-655 (1999), the teachings of which are herein incorporated by reference in their entirety.

For some methods, it may be useful to use composite matrices containing a mixture of two or more matrix forming materials. An example is the composite acrylamide-agarose gel. These gels typically contain from 2-5% acrylamide and 0.5%-1% agarose. In these gels the acrylamide provides the chief sieving function, but without the agarose, such low concentration acrylamide gels lack mechanical strength for convenient handling. The agarose provides mechanical support without significantly altering the sieving properties of the acrylamide. In such cases, the nucleic acid can be attached to the component that confers the sieving function of the gel, since that component makes the most intimate contacts with the solution phase nucleic acid target.

For many applications, gel-forming matrices such as agarose and cross-linked polyacrylamide will be preferred. However, for capillary electrophoresis (CE) applications it is convenient and reproducible to use soluble polymers as electrophoretic matrices. Examples of soluble polymers that have proven to be useful for CE analyses are linear polymers of polyacrylamide, poly(N,N-dimethylacrylamide), poly(hydroxyethylcellulose), poly(ethyleneoxide) and poly(vinylalcohol) as described in Quesada (Current Opinion in Biotechnology, vol. 8, pp.82-93, (1997)). These soluble matrices can also be used to practice the methods of the present invention. It is particularly convenient to use the methods found in the application U.S. Ser. No. 08/812,105, entitled “Nucleic Acid-Containing Polymerizable Complex” for preparation of soluble polymer matrices containing immobilized capture probes. Another approach for attaching polynucleotide probes to preformed polyacrylamide gels found in Timofeev, et al., Nucleic Acids Res., 24, 3142-3148, (1996), can also be used to attach capture probes to prepolymerized soluble linear polyacrylamide.

Nucleic acids may be attached to particles which themselves can be incorporated into electrophoretic matrices. The particles can be macroscopic, microscopic, or colloidal in nature. (See Polyciences, Inc., 1995-1996 particle Catalog, Warrington, Pa.). Cantor, et al., U.S. Pat. No. 5,482,863 describes methods for casting electrophoresis gels containing suspensions or particles. The particles are linked to nucleic acids using methods similar to those described above mixed with gel forming compounds and cast as a suspension into the desired matrix form.

Matrix Formats and Methods of Producing Matrices

Matrices may be configured in a variety of formats. For example, a linear gel may be formed by techniques including formation within a linear support, such as a trough or tube, where the gel is formed by polymerization within the support, alternatively, by subdividing a two-dimensional gel into a number of strips by partitions or formation of channels. With the trough, strip or channel formats, quantities of one, or more, copolymerizable capture probes can be added to the gel material, optionally in spatially defined positions, such as by spatially positioned dropper techniques, either before or during gel polymerization to provide one, or more, capture probes within the polymerized gel. With the tube format, a sequence of gel monomers and mixtures of gel monomers and polymerizable capture probes may be introduced into the tube sequentially such as to provide a spatially distinguished set of components and concentrations which are then polymerized in situ to preserve the components' spatial relationships. To preserve the integrity of the gel during polymerization induced shrinkage, the tube walls can be made of elastic material which laterally contracts during shrinkage of the gel. Alternatively, progressive polymerization may be induced from one end of the tube while adding more liquid material to the other end to compensate for shrinkage. Such progressive polymerization may be induced by means including diffusion of a polymerization catalytic agent, or by progressive application of polymerization inducing electromagnetic or other radiation from one end of the tube to the other, such as by movement of, or progressive exposure to, the radiation source. Alternatively, a linear format gel may be produced by taking a linear slice from a two-dimensional gel, or a linear core from a three-dimensional gel, produced as described below.

A two-dimensional gel may be formed by techniques including formation on a surface of a support, or formation between two support surfaces. A layer of gel monomer is applied and quantities of coplymerizable capture probes may be applied to the layer, optionally in a spatially significant manner, before or during polymerization, which are then polymerized in situ to preserve their spatial positions in the gel. Application of quantities of polymerizable capture probes may be effected by known means including positional programmable dropper techniques. Gel shrinkage during polymerization may be adjusted for by means including permitting contraction of the gap between support surfaces and by permitting lateral contraction with more material added from the side to compensate. A two-dimensional gel may be subdivided into a number of strips, by the use of partitions before, during or after gel formation, or by formation in channels, or by being sliced into narrower sections after formation.

Three-dimensional gels may be formed by a number of techniques. Multiple linear strips or two-dimensional layers may be repetitively constructed as above, each optionally containing localized capture probes, with each strip or layer being polymerized onto an underlying layer such that a three-dimensional volume results. Alternatively, a number of two-dimensional gels, optionally with capture probes localized in place, may be formed as above and assembled together to provide a three-dimensional structure.

Electrophoretic matrices useful for the methods described herein can be provided in a number of different formats. For example, the matrix can be provided in a format where its physical length significantly exceeds its breadth or depth, for example, contained within a tube or formatted as a narrow strip. Alternatively, the matrix can be provided in a format where its length and breadth significantly exceed its depth, for example, as a relatively thin layer on a surface or formatted as a slab. Alternatively, the matrix can be provided essentially as a solid body, where its length, breadth and depth are of the same order, for example, as an actual or approximately rectilinear, polygonal, spherical, ellipsoid solid or similar physical form. The electrophoretic matrix (or medium) can comprise a homogeneous or heterogeneous matrix material. Examples of suitable matrix materials include gel-forming polymers such as cross-linked polyacrylamide, agarose, starch and combinations thereof. Non-gel-forming polymers such as linear polyacrylamide, poly(N,N-dimethylacrylamide), poly(hydroxyethylcellulose), poly(ethyleneoxide) and poly(vinlyalcohol), as commonly used in capillary electrophoresis applications, can also serve as suitable matrices.

Immobilized Probes for Analysis of Hybridization Binding Reactions

A variety of capture probes can be used in the methods of the present invention. Typically, the capture probes of the present invention comprise a nucleic acid with a polynucleotide sequence substantially complementary to at least one nucleotide sequence region of one, or more, classes of adapter molecules, wherein the adapter molecules hybridize to the capture probe. The complementarity of nucleic acid capture probes need only be sufficient enough to specifically bind the adapter molecule. Probes suitable for use in the present invention comprise RNA, DNA, nucleic acid analogs, and chimeric probes of a mixed class comprising a nucleic acid with another organic component, for example, peptide nucleic acids. Capture probes can be single-stranded or double-stranded nucleic acids. Preferably, the capture probe will be modified in such a manner as to allow it to be immobilized within an electrophoretic medium, such as modifying the 5′-terminus with an acrylamide moiety (Acrydite™, Mosaic Technologies, Boston, Mass.).

As defined herein, the term “nucleic acid” includes DNA (deoxyribonucleic acid) or RNA (ribonucleic acid). Nucleic acids referred to herein as “isolated” are nucleic acids separated away from the components of their source of origin (e.g., as it exists in cells, or a mixture of nucleic acids such as a library) and may have undergone further processing. Isolated nucleic acids include nucleic acids obtained by methods known to those of ordinary skill in the art. (Ausubel, F. M., et al., (eds), Current Protocols in Molecular Biology, John Wiley & Sons (Pub.), vol. 1, ch. 2 through 4 (1991). These isolated nucleic acids include substantially pure nucleic acids by one, or a combination of, biological, chemical and recombinant methods from which nucleic acids have been isolated.

“Modified nucleic acids”, as used herein, include nucleic acids containing modified sugar groups, phosphate groups or modified bases. Examples of nucleic acids having modified bases, include, for example, acetylated, carboxylated or methylated bases (e.g., 4-acetylcytidine, 5-carboxymethylaminomethyluridine, 1-methylinosine, norvaline or allo-isoleucine). Probes containing modified polynucleotides may also be useful. For instance, polynucleotides containing deazaguanine and uracil bases can be used in place of guanine and thymine-containing polynucleotides to decrease the thermal stability of hybridized probes (Wetmur, Critical reviews in Biochemistry and Molecular Biology, vol. 26, pp. 227-259 (1991)). Similarly, 5-methylcytosine can be substituted for cytosine if hybrids of increased thermal stability are desired (Wetmur, Critical reviews in Biochemistry and Molecular Biology, vol. 26, pp. 227-259 (1991)). Modifications to the ribose sugar group, such as the addition of 2′-O-methyl groups can reduce the nuclease susceptibility of immobilized RNA probes (Wagner, Nature, vol. 372, pp. 333-335 (1994)). Modifications that remove negative charge from the phosphodiester backbone can increase the thermal stability of hybrids (Moody et al. Nucleic Acids Res., vol. 17, pp.4769-4782 (1989); Iyer et al. J. Biol. Chem., vol. 270, pp.14712-14717 (1995)).

As defined herein, “substantially complementary” means that the polynucleotide sequence of the capture probe need not reflect the exact polynucleotide sequence of the adapter molecule, but must be sufficiently complementary in order to hybridize with the adapter molecule under specified conditions. For example, non-complementary bases, or additional polynucleotides can be interspersed in sequences provided that the sequences have sufficient complementary bases to hybridize therewith. Generally, the degree of complementarity required is from about 90 to about 100%.

Specified conditions of hybridization can be determined empirically by those of ordinary skill in the art. For example, conditions of stringency should be chosen that significantly decrease non-specific hybridization reactions. Stringency conditions for nucleic acid hybridizations are explained in, for example, Current Protocols in Molecular Biology, Ausubel, F. M., et al., eds., vol. 1, suppl, 26, 1991, the teachings of which are herein incorporated by reference in their entirety. Factors such as probe length, base composition, percent mismatch between the hybridizing sequences, temperature and ionic strength influence the stability of nucleic acid hybrids. Stringent conditions, for example, moderate, or high stringency, can be determined empirically, depending in part on the characteristics of the probe and adapter molecule.

The length of a capture probe will be at least 5 nucleotides in length, usually between 5 and 50 nucleotides, and can be as long as several thousand bases in length. Typically, the capture probes immobilized in the universal capture gel are from about 18 to about 24 nucleotides in length.

“Nucleic acid analogs” as used herein include molecules which lack sugar-phosphate backbones, but retain the ability to form complexes via basepairing. Such nucleic acid analogs are known to those of ordinary skill in the art. Nucleic acid analogs can also be useful as immobilized probes. One example of a useful nucleic acid analog is peptide nucleic acid (PNA), in which standard DNA bases are attached to a modified peptide backbone comprised of repeating N-(2-aminoethyl)glycine units (Nielsen et al., Science, vol. 254, pp. 1497-1500 (1991)). The peptide backbone is capable of holding the bases at the proper distance to base pair with standard DNA and RNA single strands. PNA-DNA hybrid duplexes are much stronger than equivalent DNA-DNA duplexes, probably due to the fact that there are no negatively charged phosphodiester linkages in the PNA strand. In addition, because of their unusual structure PNAs are very resistant to nuclease degradation. For these reasons, PNA nucleic acid analogs are useful for immobilized probe assays. It will be apparent to those of ordinary skill in the art that similar design strategies can be used to construct other nucleic acid analogs that will have useful properties for immobilized probe assays.

Detection Schemes

Detection of the specific binding reaction, for example, detection of the immobilized adapter/target complex bound to the capture probe, can be accomplished in a number of different ways.

The target molecule can be detectably labeled prior to the hybridization reaction with the adapter molecule. Suitable labels for direct target labeling can be intensely absorbing (e.g., brightly colored), radioactive, fluorescent, phosphorescent, chemiluminescent or catalytic. Direct target labeling of nucleic acid samples using modified polynucleotides can be accomplished by a number of enzymatic methods well known to those practiced in the art (reviewed in Sambrook, et al., “Molecular Cloning: A Laboratory Manual”, 2nd edition, Cold Spring Harbor Press, Cold Spring Harbor, N.Y. 1989).

Alternatively, the target molecule can be labeled indirectly using a ligand which can be recognized by a second specific binding entity which is either labeled itself or can produce a detectable signal.

An example of such an indirect system is labeling using biotinylated polynucleotides. In this system, the sample is labeled enzymatically using standard nucleic acid labeling techniques and biotinylated polynucleotides. The resulting biotin-modified nucleic acids can be detected by the biotin-specific binding of streptavidin or avidin protein molecules. The streptavidin or avidin molecules can be conjugated to fluorescent labels, such as fluorescein or reporter enzymes, such as alkaline phosphatase or horseradish peroxidase, which can be used to produce chemiluminescent or calorimetric signals with appropriate substrates (for review see Keller and Manak, “DNA Probes”, 2nd ed., Macmillan Publishers, London, 1993; Pershing, et al., eds “Diagnostic Molecular Microbiology: Principles and Applications”, American Society for Microbiology, Washington, D.C., 1993).

Another useful detection system is the digoxigenin system which uses an antidigoxigenin antibody, conjugated to alkaline phosphatase, which recognizes digoxigenin-dUTP incorporated into nucleic acids. (Current Protocols in Molecular Biology, ed. Ausubel, F. M., vol.1, §§3.18.1 to 3.19.6, (1995), the teachings of which are incorporated herein by reference in its entirety).

Detectably labeled hybridization probes can also be used as indirect target labels. For example, target nucleic acids can be indirectly labeled prior to electrophoresis by hybridization with a detectably labeled probe, hereafter termed a “sandwich” probe. The sandwich probe is designed to hybridize with a region of the target which does not overlap the region recognized by an appropriate adapter molecule. The sandwich probe is designed to remain associated with the target during electrophoresis, and cannot bind directly to the capture probe nor to the adapter molecule.

Sandwich probes can also be used to label target molecules after electrophoretic capture. In this labeling strategy, the unlabeled target molecule is subjected to electrophoresis in complex with an appropriate adapter molecule and the adapter/target hybridizes to the capture probe first. Then, the sandwich probe is subjected to electrophoresis through the capture layer. In effect, the captured adapter/target complex now acts as a new “capture” probe for the sandwich probe. The captured target sandwich probe complex can now be detected through the sandwich probe label.

Blotting techniques can also be adapted for detection of target bound capture probes. For example, a detection surface is juxtaposed to the separation medium having bound sample components, and the sample components then migrate to the detection surface, optionally assisted by, for example, chemical means such as solvent or reagent changes, where the transferred sample components are detected by known means such as optical detection of intercalating dyes, or by detection of radioactivity from hybridized radioactive species, or other known means.

A variety of optical techniques can be used to detect the presence of sample components bound to the capture probes. For example, if the capture probes are arranged in a linear array, the position and intensity of each signal may be measured by mechanically or optically scanning a single detector along the array of detectable signals. Alternatively, a linear array of detectable signals may be detected by a linear array detector, such as by juxtaposition of the array detector to the array of detectable signals or by optically imaging all or part of the signal array onto the array detector.

When the capture probes with detectable signals are arranged as a two-dimensional array, a number of detection schemes may be employed. A single detector may be used to measure the signal at each point by mechanical or optical scanning, or by any combination. Alternatively, a linear optical detection array may be used to detect a set of signals by juxtaposition or optical imaging, and multiple sets of such signals may be detected by mechanically or optically scanning the signal array or detector. Alternatively, the two-dimensional array of capture probes may be optically detected in whole or in part by a two-dimensional optical area detector by juxtaposition to, or optical imaging of, the array of optical signals from the immobilized capture probes.

When the capture probes are arranged as a three-dimensional array, detection of individual signals may be arranged by the above techniques, optionally assisted by first physically taking one, or more, sub-sections of the array. Alternatively, optical schemes such as confocal microscopic techniques may be employed whereby one or a number of detectable signals are imaged and detected with minimal interference from others, and other signals are subsequently detected after optical adjustment.

Methods of Universal Capture Gel Use

The methods of the present invention are applicable to analysis of any chemical entity that can be subjected to electrophoresis (e.g., a charged molecule that has detectable mobility when placed in an electrophoretic field) and that binds to, or is bound by, nucleic acids. Such entities include, for example, DNA or RNA samples, nucleic acid binding proteins, nucleic acid analogs, modified nucleic acids, and aptamer binding partners (aptamers are nucleic acids that are selected to bind to specific binding partners such as peptides, proteins, drugs, polysaccharides and small organic molecules, for example, theophylline and caffeine; Jenison, et al., Science, 263:1425-1429 (1994)). For example, methods described herein can be used for analysis and purification of target nucleic acids using immobilized capture probes, where specific binding involves base pairing interactions between sample nucleic acids and the capture probe, as in nucleic acid hybridization. The methods described herein are also useful for purification of sequence-specific nucleic acid binding proteins, since synthetic nucleic acids of defined sequence can be immobilized in matrices commonly used for protein electrophoresis.

The sample containing the adapter/target complex can be detectably labeled before, during, or after the electrophoresis step. The adapter can be labeled prior to hybridizing with the target molecule. Alternatively, the target molecule can be labeled prior to hybridization with the adapter molecule. Also, both can be labeled prior to hybridization with each other. Detecting the presence of adapter/target/capture probe complexes immobilized within the matrix is indicative of the presence of the target molecule that is bound by the capture probe via an adapter molecule. This in turn can be interpreted as the presence of some microbe, for example, in a test sample; the identification of one, or more, mutations in a nucleic acid molecule, for example, in a certain strain of bacteria or virus; or the identification of one, or more, particular bacterial species in, for example, a culture. Once a test sample containing an adapter/target complex is introduced into the electrophoretic medium it is subjected to an electrical field resulting in the electrophoretic migration of the test sample through the matrix, under conditions and time sufficient for the adapter/target complex, of the test sample, if present, to bind to one, or more, capture probes, resulting in adapter/target/capture probe complexes immobilized in the matrix. Typical voltage gradients used in nucleic acid electrophoresis procedures range from approximately 1 V/cm to 100 V/cm. Other field strengths can be useful for certain highly specialized applications.

In one embodiment of the invention, a method for detecting a target molecule contained in a test sample using a universal capture gel is disclosed. An adapter/target hybridization complex is formed by mixing an appropriate adapter molecule with a test sample containing a target molecule under conditions suitable for hybridization. An appropriate adapter molecule is an adapter molecule comprising at least one nucleotide sequence region complementary to at least one nucleotide sequence region contained within a target molecule. The adapter molecule comprises at least two defined nucleotide sequence regions. First, the target-specific region is complementary to a nucleotide sequence region contained within a target molecule. This hybridization product, that is, the adapter/target complex, can then be introduced into the universal capture gel. The universal capture gel contains capture probes which are immobilized within the medium. The universal capture gel is subjected to an electric field resulting in the electrophoretic migration of the hybridization complex (adapter/target complex) formed above. When the adapter/target complex comes into contact with an appropriate capture probe, that is, a capture probe that is complementary to the capture probe-specific nucleotide sequence region (the second defined nucleotide sequence region of an adapter molecule) of the adapter probe, then the adapter/target complex will become immobilized within the medium through the binding of the adapter molecule to the immobilized capture probe. Detection of the new tripartite complex formed by the adapter/target/capture probe complex is indicative of the presence of the target molecule in the test sample.

In another embodiment, a method of detecting one, or more, target molecules in a test sample using a universal capture gel comprising only one class of immobilized capture probes is disclosed. Using this universal capture gel, multiple target molecules can be detected. Multiple classes of adapter molecules can be used wherein they all share one nucleotide sequence region that is complementary to the capture probe immobilized in the electrophoretic medium. The classes of different adapter molecules differ in that their target-specific nucleotide sequence region is specific for a particular target molecule contained within a test sample. In this embodiment it may not be necessary to isolate the different target molecules in discrete regions of the gel, but only necessary to qualitatively detect the presence of any of these particular target molecules in the test sample and therefore the capture probes can be immobilized throughout the gel.

Alternatively, the immobilized capture probes can be immobilized in discrete regions of the electrophoretic medium. The adapter molecules used are first mixed with target molecules from the test sample under conditions suitable for hybridization wherein, the adapter molecule specific for a particular target molecule will hybridize to that target molecule. This hybridization step typically proceeds until all, or any desired number of target molecules, have hybridized to their complementary adapter molecule. Then the hybridization complex preparation containing one, or more, adapter/target complexes, is subjected to electrophoresis through the universal capture gel. The adapter/target complexes will migrate in the electrophoretic medium until they come in contact with an immobilized capture probe which is specific for the adapter molecule. Once the adapter/target complex comes into contact with an immobilized capture probe a second complex is formed between the adapter/target complex and immobilized probe forming an adapter/target/capture probe tripartite complex. This tripartite complex is immobilized to the electrophoretic medium via the immobilized capture probe. The detection of this tripartite complex is indicative of the presence of the target molecule, or molecules, in the test sample.

In still another embodiment, a method for detecting one, or more, target molecules from a test sample using a universal capture gel using multiple classes of capture probes is disclosed. Preferably, in this embodiment multiple classes of capture probes are immobilized within the electrophoretic medium in discrete regions. This allows for the isolation and identification of one target molecule from another captured in this embodiment. Alternatively, the capture probes can be immobilized throughout the electrophoretic medium. Preferably, however, one or more classes of capture probes are immobilized in discrete regions of the gel. Preferably, only one class of capture probe is immobilized to any given discrete capture layer within the gel. A capture layer is a layer comprising immobilized capture probes within the electrophoretic medium.

Multiple adapter molecules are used which have a complementary nucleotide sequence region to a particular class of capture probe immobilized in the electrophoretic medium.

An adapter molecule corresponding to a particular class of immobilized capture probe has a target-specific nucleotide sequence region complementary to a specific target molecule. The adapter molecules and target molecules are first mixed with one another under conditions suitable for hybridization. This first step continues until all, or any desired number of target molecules are hybridized to its corresponding complementary adapter molecule. These adapter/target complexes are then subjected to electrophoresis. When the individual adapter/target complex comes in contact with the appropriate immobilized capture probe, then the complex becomes immobilized to the gel. The location of the immobilized tripartite complex of adapter/target/capture probe depends upon the specificity between the adapter molecule (complexed with a particular target molecule) and the capture probe immobilized in a particular, discrete region of the gel.

For example, there can be five target molecules to be analyzed using one universal capture gel. This universal capture gel may comprise five discrete capture layers containing five separate classes of immobilized capture probes specific for their particular adapter molecule. Five classes of adapter molecules are used where each class is specific for a particular class of capture probe as well as being specific for a particular target molecule. Once the adapter molecules are mixed with the target molecules of the test sample, then five sets of adapter/target complexes are formed and can be subjected to electrophoresis. Each adapter/target complex will migrate in the electrophoresis medium until it comes in contact with the appropriate immobilized capture probe forming an immobilized tripartite complex. Therefore, there could be five separate capture layers comprising five different tripartite complexes, indicating that the test sample had at least five different classes of target molecules.

One-Dimensional Arrays for Analysis of Target Molecules

In this embodiment of the present invention, a sample containing a target molecule is subjected to clectrophoresis through a series of discrete matrix layers each of which contain at least one class of capture probes. Alternatively, the capture probes can be throughout the matrix. For example, in a hybridization binding reaction, a target nucleic acid that is bound to an adapter molecule that is complementary to the capture probe can hybridize to that capture probe which is contained within a gel layer and is thus retained in the gel layer, that is, the capture layer. Noncomplementary sample nucleic acids pass through the capture layer. The presence of hybrids between capture probes and complementary adapter/target complex is detected within the capture layer by appropriate labeling strategies described herein.

There are several important advantages to this one-dimensional format. First, all of the sample containing one, or more, adapter/target molecule complexes pass through the capture layer, and is therefore available for hybridization. This is a major advantage over most other solid phase hybridization methods. Using high concentrations of immobilized probe, it is possible to capture all hybridizable adapter/target complexes in a small gel band.

Second, intact nucleic acid species that have discrete electrophoretic mobilities are not required for analysis by this method. Since the use of specific adapter polynucletides convey specificity with respect to the target molecule. In traditional zonal electrophoresis, all target molecules must migrate as a discrete band for detection.

Third, the sample volume is not important. In the present invention, all target molecules that are in complexes with their appropriate adapter molecules pass through the capture layer even though large sample volumes are used. This is a significant advantage over traditional zonal electrophoresis, where the sample volume needs to be as small as possible for maximum detection sensitivity and resolution.

In this embodiment, the capture layer can contain single or multiple classes of capture probes. The use of multiple capture probes in a single layer is useful for assays where any one of a number of different organisms need to be detected.

Multiple capture layers can be also be used in this embodiment. It is straightforward to cast multiple capture layers sequentially in the same gel apparatus to create a multiplex hybridization assay. During the assay, the target molecule in a complex with an appropriate adapter molecule is subjected to electrophoresis through all of the layers, and complementary adapter molecules are captured at each layer by an immobilized capture probe specific for that particular class of adapter molecule. The amount of hybrid in each layer directly reflects the sample composition with respect to the capture probes used.

Conditions can be identified to ensure that only properly hybridized adapter/target molecule complexes will be retained in each layer. Electrophoretic hybridization with capture probes as long as twenty bases can be carried out using traditional non-denaturing gels and buffer systems at room temperature. Fully complementary hybrids of this size appear to be stable for many hours. However, additional stringency can be achieved by adding denaturants such as urea or formamide to the gel, or running the gel at elevated temperatures.

Two Dimensional Probe Arrays

One dimensional probe arrays can be used for analysis that employ limited numbers of capture probes. For analysis of larger numbers of sequences, a two-dimensional array of immobilized probes can be used. The arrays can be formed in a number of ways. Simple two-dimensional arrays can be cast, for example, in conventional slab gel devices using multiple vertical aligned spacers, in effect creating an array of one dimensional arrays.

More complex two dimensional arrays can be created in two steps, first, polymerizing the capture probe regions as an array of matrix (for example, polyacrylamide gel) dots on one plate, then “sandwiching” the dots by placing an upper gel plate over the array and filling in the empty spaces between the probe dots with unmodified gel.

In either case, the sample containing adapter/target complexes is loaded as a band across the entire length of the matrix, for example, at the top edge of the matrix. In this embodiment, the entire test sample does not contact all of the capture probes. However, for most applications where two-dimensional analysis is desirable, such as library screening or gene expression analysis, the sample nucleic acids are present at high copy numbers, and so this problem does not present a significant obstacle.

Three-Dimensional Probe Arrays

The hybridization methods described herein may also encompass three-dimensional arrays, such as may be particularly useful for multiplexed parallel assays, for example, high throughput and/or cost-effectiveness. Such assays may be provided in the format of three-dimensional solids, where multiple samples containing adapter/target complexes may be applied to a surface or face, then made to migrate through the volume of the solid such that one, or more, regions of capture probe are encountered. The array may be produced such that each sample encounters the same sequence of capture probes during migration through the array, alternatively, different sequences of capture probes may be positioned for this purpose, such as to analyze different test sample mixtures or to analyze differing sets of components within one, or more, test sample mixtures.

Sample Purification/Concentration by Hybridization with Immobilized Probes

The electrophoresis methods described herein are especially useful for selectively purifying specific target molecules from a crude, or semi-crude, mixture. For example, a semi-purified mixture (perhaps using the supernatant preparation) of cell extract mixed with an appropriate adapter, or adapters, is placed over a gel containing an imunobilized capture probe. The mixture undergoes electrophoresis through the gel. Target molecules are immobilized on the layer containing the capture probes through binding with the appropriate adapter molecule. Non-target molecules with the same charge as the targets are attracted to the electrode of opposite electrical polarity (which will be referred to here as the “attracting electrode”) and pass through the capture probe layer, eventually electrophoresing out of the gel. Non-target molecules of the opposite charge migrate out of the sample well toward the non-attracting electrode. Uncharged sample molecules remain in the sample well and do not enter the gel. After allowing sufficient electrophoresis time, in order to be sure that all charged non-target molecules have been removed from the gel, the captured target molecules are eluted from the gel by one of two methods:

1) Continued electrophoresis under denaturing conditions (e.g., by raising the temperature of the matrix or increasing electrophoretic voltage).

2) Continued electrophoresis after chemical or photochemical cleavage of the chemical linkage between the capture probe and the matrix.

The eluted target molecules can be concentrated and recovered from the attracting electrode chamber by several methods. For instance, after undergoing electrophoresis, some non-target sample molecules will migrate out of the gel, some non-target molecules will migrate to the attracting electrode chamber which can be flushed out with an appropriate wash solution, finally, the target molecules can be eluted directly into the original attracting electrode chamber. Alternatively, the electrophoresis device can have two attracting electrodes so that one is used to clear the non-target components from the sample while the second is used to elute the target molecules. Alternatively, the electrophoresis device can be constructed with a replaceable attracting electrode chamber so that after removal of non-target components, the attracting electrode chamber can be replaced with a clean chamber in order to perform target elution.

This embodiment is particularly well-suited for purification of specific nucleic acids from semi-crude biological samples by hybridization methods. By employing the appropriate adapter molecule, specific target molecules can be isolated from a semi-crude preparation. First, these methods result in the capture of substantially all sample nucleic acids with the desired sequence because the whole test sample comprising adapter/target complexes must pass through the capture zone, also the concentration of the capture probe can be made arbitrarily high which ensures capture success.

Second, these methods result in substantial purification of target nucleic acids in one step because charged sample contaminants are eliminated during electrophoresis and uncharged contaminants are eliminated since they cannot enter the matrix.

Third, very large samples can be used. The nucleic acids undergo electrophoresis in free solution in virtually the same manner as they do in polymeric matrices. Therefore, large sample volumes can be used. The matrix layer acts like a highly selective filter to select only the desired nucleic acids (formed in a complex with an appropriate adapter molecule) from the sample.

Fourth, large volumes of very dilute samples can be concentrated quantitatively using the methods described herein.

In one embodiment of the present invention, target molecules are purified by employing modified adapter molecules. Once a tripartite hybridization complex comprising an adapter molecule/target molecule/universal capture probe is formed a modified adapter molecule can be used to displace either the target molecule alone, or the target/adapter complex from immobilization to the gel matrix. In order to displace the target molecule from the tripartite complex, a modified adapter molecule comprising a nucleotide sequence region that is complementary to the unmodified adapter's target-specific nucleotide sequence region. With a molar excess of from about 2 to about 5 fold of a modified adapter molecule, displacement of the target molecule from the complex can be effectuated by subjecting the modified adapter molecule to electrophoresis. When the modified adapter molecule comes into contact with the tripartite complex, the modified adapter molecule will displace the target molecule from the tripartite complex and will bind to the adapter molecule hybridized to the immobilized capture probe. The target molecule can then continue to migrate through the gel and can be retrieved.

In another embodiment, a modified adapter molecule can comprise a nucleotide sequence region that is complementary to the unmodified adapter's capture probe-specific nucleotide sequence region. This modified adapter molecule can be placed into an electrophoresis medium already containing a tripartite hybridization complex (comprising an adapter molecule/target molecule/universal capture probe). The modified adapter molecule can be subjected to electrophoresis and migrate until it comes into contact with the tripartite complex. The modified adapter can displace the adapter/target complex form the immobilized capture probe and hybridize to the capture probe. The released adapter/target complex can continue to migrate through the gel.

The features and other details of the invention will now be more particularly described and pointed out in the examples. It will be understood that the particular embodiments of the invention are shown by way of illustration and not as limitations of the invention. The principle features of this invention can be employed in various embodiments without departing from the scope of the invention.

Exemplification

### Detecting Target RNA Using an Adapter Molecule

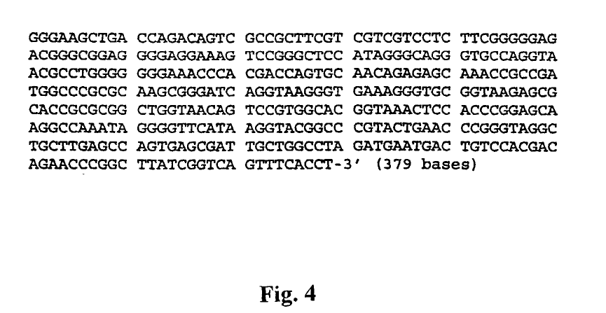

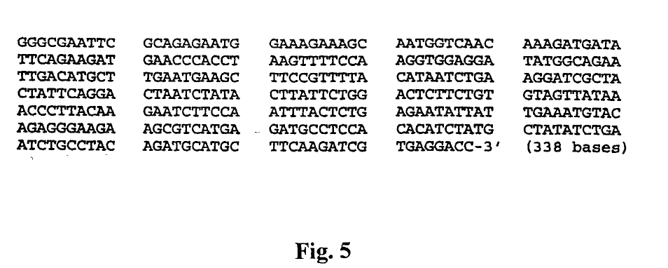

The experiment was performed using two different and unrelated RNA target molecules referred to as RNA 1 and RNA 2 produced using T7 RNA polymerase-based in vitro transcription kit, Promega Corp., Madison, Wis., supplemented with fluorescent nucleotide as label, fluorescein-rUTP, Boehringer Mannheim, Indianapolis, Ind.). In this experiment the RNA molecules are labeled with fluoroscein. RNA 1 and RNA 2 (approximately 0.5 to 2 pmoles) were mixed, independently, with a specific adapter molecule. The adapter for RNA 1, which is highly folded forming a higher ordered structure) was AdRNP13V-128: 5′-CGG TTT GCT CTC TGT TGC ACT GTG AAT ACG TTC CCG GGC CT-3′ [SEQ ID NO. 1]. The adapter for RNA 2 was UNMB2: 5′-CTT AGG TGG GTT CAT CTT CTG GTG AAT ACG TTC CCG GGC C-3′ [SEQ ID NO. 2]. RNA 1 was transcribed from a PCR product containing the E. coli M1 RNA gene. (See FIG. 4, SEQ ID NO. 3). RNA 2 was transcribed from a plasmid containing a gene fragment from the human nonmuscle mysosin B gene cloned into pGEM3Zf(−), Promege, Madison, Wis. (See FIG. 5, SEQ ID NO. 4). This vector contains a T7 RNA polymerase promoter that was used to produce RNA 2 in an in vitro transcription reaction, as described above for RNA 1. Increasing amounts of the appropriate adapter molecule were added to separate reaction vials to a final volume of 20 μL in 0.1 M NaCl. The samples were heated to 90° C. and allowed to cool to room temperature. Approximately 4 μL of a ficoll sample loading buffer containing xylene cyanol and bromophenol blue were added to each sample and then 12 μL were loaded into the gel.

The gel was 5% polyacrylamide (29:1, acrylamide:bis) and 0.5× TBE. A capture layer containing 13V acrydite polynucleotide capture probe (10 μM, total volume of 600 μL) was placed in the gel about 1 cm below the sample wells. The capture probe sequence used was: 5′-acrylamide-AGG CCC GGG AAC GTA TTC AC-3′ [SEQ ID NO. 5]. The sample were subjected to electrophoresis using 200 V for 35 minutes at room temperature.

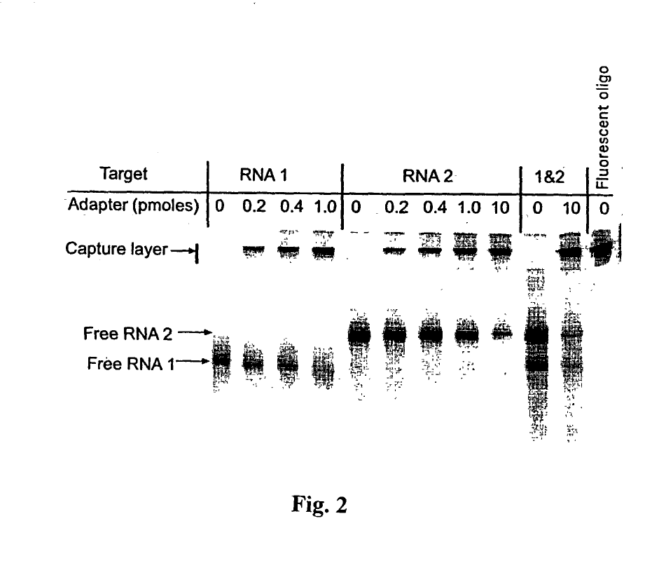

The gel was then analyzed using a Molecular Dynamics fluorimager (Molecular Dynamics). FIG. 2 shows the results of this experiment. The results demonstrated that when there was no adapter present in the reaction mixture described above, there was no capture of the target RNA, i.e., all of the target RNA migrates through the capture layer (were the immobilized capture probes reside) and gives a band towards the bottom of the gel. In contrast, when the adapter is present a sharp fluorescent band is detected at the top surface of the capture layer. As the amount of the adapter increases so does the relative percentage of target RNA become captured. The results also demonstrate that a heterogenous population of target molecules can be subjected to this invention resulting in their capture in one homogenous layer, or in layers where the respective adapter is specific for its capture probe (there can be multiple capture layers specific for individual adapter molecules).

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A universal gel comprising a universal capture probe comprising a nucleotide sequence region complementary to a capture probe-specific nucleotide sequence region contained within an adapter molecule, wherein said capture probe is covalently immobilized within a medium suitable for electrophoresis.

2. The universal gel of claim 1, wherein said universal capture probe comprises SEQ ID No. 5.

3. The universal gel of claim 2, wherein an adapter molecule is selected from the group consisting of: SEQ ID NOS. 1 and 2.

4. A universal capture gel system comprising:
(a) a universal capture probe, wherein said universal capture probe comprises a nucleotide sequence region complementary to said adapter molecule;
(b) an adapter molecule, wherein said adapter molecule comprises a capture probe-specific nucleotide sequence region which is complementary to a nucleotide sequence region contained within said universal capture probe, and a target-specific nucleotide sequence region complementary to a nucleotide sequence region contained within a target molecule; and
(c) a medium suitable for electrophoresis, wherein said universal capture probe is covalently immobilized within said medium suitable for electrophoresis.

5. A universal gel hybridization complex comprising:
(a) at least one adapter molecule comprising a capture probe-specific nucleotide sequence region that is complementary to a nucleotide sequence region contained within a universal capture probe, and a target-specific nucleotide sequence region that is complementary to a nucleotide sequence region of at least one target molecule; and
(b) at least one target molecule comprising a nucleotide sequence complementary to said adapter's target-specific nucleotide sequence region of (a), wherein the adapter molecule of (a) is hybridized with the target molecule of (b) thereby forming a hybridization complex.

6. The method of claim 5, wherein said universal capture probe is SEQ ID No. 5.

7. The universal gel hybridization complex of claim 5, wherein said adapter molecule contains from about 10 to about 100 nucleotides.

8. The universal gel hybridization complex of claim 5, wherein said adapter molecule is either a single-stranded or double-stranded nucleic acid.

9. The adapter molecule of claim 8, wherein said nucleic acid is either DNA or RNA.

10. The universal gel hybridization complex of claim 5, wherein said adapter molecule is an aptamer.

11. The universal gel hybridization complex of claim 5, wherein said target molecule is selected from the group consisting of: nucleic acids, nucleic acid analogs, modified nucleic acids, aptamer binding partners and nucleic acid binding proteins.

12. The universal gel hybridization complex of claim 5, wherein said target molecule is either a single-stranded or double-stranded nucleic acid, modified nucleic acid, or nucleic acid analog molecule.

13. The universal gel hybridization complex of claim 5, wherein said target molecule is ribonucleic acid.

14. The universal gel hybridization complex of claim 5, wherein said target molecule is deoxyribonucleic acid.

15. The universal gel hybridization complex of claim 5, wherein said target molecule contains from about 10 to about 100,000 nucleotides.

16. The universal gel hybridization complex of claim 5, wherein the test sample comprises a target molecule selected from the group consisting of: bacterial molecules, viral molecules, fungal molecules, parasitic molecules, plant molecules, animal molecules and combinations thereof.

17. A method of detecting the presence, or absence, of a target molecule in a test sample, comprising the following steps:
(a) forming a hybridization complex by contacting:
(i) an adapter molecule comprising a capture probe-specific nucleotide sequence region that is complementary to a nucleotide sequence region contained within at least one universal capture probe polynucleotide immobilized within an electrophoresis medium, and a target-specific nucleotide sequence region that is complementary to a nucleotide sequence region contained within at least one target molecule; and
(ii) the test sample wherein the test sample contains a target molecule comprising a nucleotide sequence complementary to said adapter's target-specific nucleotide sequence region, under conditions suitable for hybridization of said adapter molecule with said target molecule;
(b) introducing said hybridization complex of (a) into an electrophoresis medium comprising an immobilized universal capture probe wherein the capture probe comprises a nucleotide sequence region complementary to said adapter's capture probe-specific nucleotide sequence region;
(c) subjecting said electrophoresis medium to an electric field resulting in the electrophoretic migration of the hybridization complex formed in (a) into at least one region of said medium containing at least one class of immobilized universal capture probes, thereby forming an adapter/target/universal capture probe complex; and
(d) detecting said adapter/target/universal capture probe complex, wherein the detection of said adapter/target/universal capture probe complex is indicative of the presence of at least one target molecule within said test sample.

18. The method of claim 17, wherein the electrophoretic medium used in said universal gel is selected from the group consisting of: a polyacrylamide polymer, an agarose polymer, a starch polymer and a combination thereof.

19. The method of claim 17, wherein said target molecule is labeled.

20. The method of claim 17, wherein said label is selected from the group consisting of: radioactivity, catalytic, chemiluminescent, phosphorescent, fluorescent and luminescent.

21. The method of claim 17, wherein said universal capture probes are immobilized within at least one discrete region of the electrophoresis medium.

22. The method of claim 17, wherein said universal capture probes are immobilized throughout said electrophoresis medium.

23. The method of claim 17, wherein said universal capture probe is selected from the group consisting of: nucleic acids, nucleic acid analogs, and modified nucleic acids.

24. The method of claim 17, wherein said universal capture probe is SEQ ID NO. 5.

25. The method of claim 17, wherein said universal capture probe contains from about 5 to about 100 nucleotides.

26. The method of claim 17, wherein said target molecule is selected from the group consisting of: nucleic acids, nucleic acid analogs, modified nucleic acids, aptamer binding partners and nucleic acid binding proteins.

27. The method of claim 17, wherein said target molecule is either a single-stranded or double-stranded nucleic acid, modified nucleic acid, or nucleic acid analog molecule.

28. The method of claim 17, wherein said target molecule is ribonucleic acid.

29. The method of claim 17, wherein said target molecule is deoxyribonucleic acid.

30. The method of claim 17, wherein said target molecule contains from about 10 to about 100,00 nucleotides.

31. The method of claim 17, wherein the test sample comprises a target molecule selected from the group consisting of: bacterial molecules, viral molecules, fungal molecules, parasitic molecules, plant molecules, animal molecules and combinations thereof.

32. A method of detecting the presence or absence of a target molecule in a test sample, comprising the following steps:
(a) introducing said test sample into an electrophoretic medium comprising immobilized capture probes;
(b) subjecting said electrophoretic medium of (a) to an electric field resulting in the electrophoretic migration of target molecules of the test sample into said electrophoretic medium;
(c) introducing an adapter molecule into said electrophoretic medium of (a), wherein said adapter is subjected to electrophoretic migration through said electrophoretic medium, wherein said adapter molecule comprises an electrophoretic mobility greater than said target molecule;
(d) contacting said adapter with said target molecule within said electrophoretic medium, thereby forming an adapter/target complex;
(e) contacting said adapter/target complex of (d), with said immobilized capture probe, thereby forming an adapter/target/capture probe complex; and
(f) detecting said adapter/target/universal capture probe complex of (e), wherein the detection of said adapter/target/universal capture probe complex is indicative of the presence of at least one target molecule within said test sample.

33. A method of detecting the presence or absence of a target molecule in a test sample, comprising the following steps:
(a) introducing an adapter molecule into an electrophoretic medium comprising immobilized capture probes;
(b) subjecting said electrophoretic medium to an electric field resulting in the electrophoretic migration of said adapter molecule into said medium comprising immobilized capture probes, thereby forming an adapter/capture probe complex;
(c) introducing said test sample into said electrophoretic medium of (b), wherein target molecules of the test sample are subjected to electrophoretic migration into said electrophoretic medium, thereby forming an adapter/target/capture probe complex; and
(d) detecting said adapter/target/capture probe complex of (c), wherein the detection of said adapter/target/capture probe complex is indicative of the presence of at least one target molecule within said test sample.

34. A method of detecting the presence, or absence, of one, or more, target molecules in a test sample, comprising the following steps:
(a) forming multiple hybridization complexes by contacting:
(i) appropriate adapter molecules, wherein each adapter molecule comprises a capture probe-specific nucleotide sequence region that is complementary to a nucleotide sequence region contained within a universal capture probe immobilized within an electrophoresis medium, and a target-specific nucleotide sequence region that is complementary to a nucleotide sequence region contained within a specific target molecule; and
(ii) one or more target molecules, wherein each target molecule comprises a nucleotide sequence complementary to an adapter's target-specific nucleotide sequence region, under conditions suitable for hybridization of said adapter molecule said target molecule;
(b) introducing said hybridization complexes of (a) into an electrophoretic medium comprising universal capture probes immobilized within one, or more, discrete regions of said medium;
(c) subjecting said electrophoretic medium to an electric field resulting in the electrophoretic migration of the hybridization complexes formed in (a) into at least one region of said universal gel containing only one class of universal capture probes immobilized in one, or more, discrete regions of said universal gel, thereby forming adapter/target/universal capture probe complexes; and
(d) detecting said adapter/target/universal capture probe complex, wherein the detection of said adapter/target/universal capture probe complex is indicative of the presence of at least one target molecule within said test sample.

35. A method of purifying a target molecule from a test sample, comprising the following steps:
(a) forming a hybridization complex by contacting:
(i) an adapter molecule comprising a capture probe-specific nucleotide sequence region that is complementary to a nucleotide sequence region contained within at least one universal capture probe polynucleotide immobilized within an electrophoresis medium, and a target-specific nucleotide sequence region that is complementary to a nucleotide sequence region contained within at least one target molecule; and
(ii) the test sample wherein the test sample contains a target molecule comprising a nucleotide sequence complementary to said adapter's target-specific nucleotide sequence region, under conditions suitable for hybridization of said adapter molecule with said target molecule;
(b) introducing said hybridization complex of (a) into an electrophoresis medium comprising an immobilized universal capture probe wherein the capture probe comprises a nucleotide sequence region complementary to said adapter's capture probe-specific nucleotide sequence region;
(c) subjecting said electrophoresis medium to an electric field resulting in the electrophoretic migration of the hybridization complex formed in (a) into at least one region of said medium containing at least one class of immobilized universal capture probes, thereby forming an adapter/target/universal capture probe complex;
(d) introducing a modified adapter molecule to said electrophoresis medium, wherein said modified adapter molecule comprises a nucleotide sequence region complementary to the unmodified adapter's target-specific nucleotide region;
(e) subjecting said modified adapter molecule to electrophoresis by applying an electric field to said electrophoresis medium; and
(f) displacing said target molecule from said adapter/target/universal capture probe complex by contacting said unmodified adapter with said adapter/target/universal capture probe complex, wherein said modified adapter displaces said target and hybridizes to said adapter molecule.

36. The method of claim 35 wherein in step (d), said modified adapter comprises a nucleotide sequence region complementary to said unmodified adapter's capture probe-specific nucleotide sequence, and wherein in step (f), the target/adapter complex is displaced from the adapter/target/universal capture probe complex by contacting said modified adapter, under conditions suitable for hybridization.
